(19) United States
(12) Patent Application Publication
(10) Pub. No.: US 2003/0162287 A1
(43) Pub. Date: Aug. 28, 2003

(54) Carrier for cell culture and method for culturing cells

(75) Inventors: Akira Yamamoto, Tokyo (JP); Ken Sugo, Saltama (JP)

Correspondence Address: GREENBLUM & BERNSTEIN, P.L.C., 1950 ROLAND CLARKE PLACE, RESTON, VA, 20191, US

(73) Assignee: PENTAX Corporation, Tokyo (JP)

(21) Appl. No.: 10/372,256

(22) Filed: Feb. 25, 2003

(30) Foreign Application Priority Data
Feb. 25, 2002 (JP) 2002-048603

(51) Int. Cl. C12M 1/00; C12N 5/02; C12M 3/00

(52) U.S. Cl. 435/289.1; 435/325

(57) ABSTRACT

A carrier for cell culture which enables cells to efficiently grow thereon is provided. A carrier 1 comprises a particulate base body 2 mainly formed of a resin material and a coating layer 3. The coating layer 3 is formed from particles 31 of a calcium phosphate-based compound, and the particles 31 are partially embedded in the base body 2 at the vicinity of the surface thereof whereby the coating layer 3 coats the surface of the base body 2 with a calcium phosphate-based compound. Such a carrier 1 enables cells to adhere to and grow on the surface thereof. Therefore, the carrier 1 can be used for cell culture, especially high-density three-dimensional cell culture. The coating layer 3 can be formed, for example, by colliding porous particles of a calcium phosphate-based compound against the surface of the base body 2.

### BACKGROUND OF THE INVENTION

1. Field of the Invention

This invention relates to a carrier for cell culture and a method for culturing cells.

2. Description of the Prior Art

Recently, cell culture technology is applied in various industries or for various fields of research. Some examples of the technology's application include cell/tissue engineering, safety studies on drugs, and production of proteins for use in the treatment and diagnosis of disease.

At present, cultivation of cells, in particular cultivation of anchorage-dependent cells, is carried out by high-density three-dimensional cell culture (suspension culture) rather than plate culture for cultivating large quantities of anchorage-dependent cells efficiently. While the plate culture is performed using a culture flask, the three-dimensional cell culture is performed using carriers that serve as scaffolds for cell growth.

In such high-density three-dimensional cell culture, carriers made of polystyrene, DEAE-cellulose, polyacrylamide or the like are used.

A problem exists with such carriers, however, in that cells may be unable to properly adhere to the carriers, and even in the case where cells have properly adhered to the carriers they may still not be able to satisfactorily grow on the carriers.

### SUMMARY OF THE INVENTION

In view of the problem stated above, it is an object of the present invention to provide a carrier for cell culture on which cells are able to efficiently grow.

To achieve the object stated above, the present invention is directed to a carrier for cell culture which enables cells to adhere to and grow on a surface thereof, the carrier comprising:

a base body having a particulate form and being mainly formed of a resin material, the base body having a surface; and

a coating layer formed from particles of a calcium phosphate-based compound, wherein the coating layer is provided on the surface of the base body, with the particles of the calcium phosphate-based compound being partially embedded in the base body at the vicinity of the surface thereof.

Such a carrier for cell culture can be formed to have a variety of properties that are required for a carrier for use in cell culture (especially, microcarrier culture).

In the present invention, it is preferred that when the maximum length of the cell to be adhered to the carrier for cell culture is defined as L1 (μm) , and the average particle size of the carrier for cell culture is defined as L2 (μm), L2/L1 is within the range of 2 to 100. By this, adhesion and growth of cells is facilitated.

Further, it is also preferred that L2 is within the range of 50 to 500 μm, by which adhesion and growth of cells is further facilitated.

Furthermore, it is also preferred that the density of the carrier for cell culture is within the range of 0.8 to 1.4 g/cm³, which makes it possible to uniformly suspend carriers for cell culture in a culture solution.

Moreover, it is also preferred that the average particle size of the base body is within the range of 50 to 500 μm, which makes it easy to obtain a carrier for cell culture having a preferred average particle size.

Moreover, it is also preferred that the density of the base body is within the range of 0.8 to 1.4 g/cm³, which makes it easy to obtain a carrier for cell culture having a preferred density.

Moreover, it is also preferred that the average thickness of the coating layer is within the range of 0.1 to 5 μm, which makes it possible to properly coat the base body and obtain a carrier for cell culture having a preferred density.

Moreover, it is also preferred that the coating layer is formed by colliding porous particles of the calcium phosphate-based compound against the surface of the base body. By doing so, it is possible to easily and reliably form the coating layer.

In such a case, it is preferred that the porous particles are manufactured by agglomerating primary particles of the calcium phosphate-based compound. By using porous particles manufactured in such a way, it is possible to more reliably coat the surface of the base body because such porous particles are effectively fragmented when collided against the base body.

The carrier for cell culture of the present invention as has been described above is particularly suitable for use in microcarrier culture that is one of various techniques for cell culture.

Further, the kind of cell to be cultured is preferably an animal cell. Animal cells can be applied in a variety of fields, and by using animal cells it is possible to effectively produce a protein having a complex structure.

Another aspect of the present invention is directed to a method for culturing cells, the method being characterized by using carriers containing the carrier for cell culture described above.

Still another aspect of the present invention is directed to a method for culturing cells, the method being characterized in that a culture solution, in which carriers containing the carrier for cell culture described above and cells are suspended, is agitated to cause the cells to adhere to the surfaces of the carriers, thereby enabling the cells to grow thereon. By culturing cells under agitation in a culture solution, it is possible to increase the efficiency of cell growth. In this method, the speed of agitation is preferably within the range of 5 to 100 rpm. By setting the speed of agitation within the above range, it is possible to further increase cell growth efficiency.

In these methods of the present invention described above, it is preferred that the carriers have been subjected to sterilization prior to use so as to reduce or eliminate deleterious effects on cells, which may otherwise occur as a result of the growth of microorganisms or molds, to thereby enable cells to more efficiently grow on the carriers.

Further, it is also preferred that the sterilization of the carriers is carried out using a sterilizing solution, by which it is possible to efficiently sterilize large quantities of carriers.

Furthermore, the sterilizing solution is preferably an alkaline solution, since such a solution has excellent sterilizing properties for destroying (reducing) microorganisms or molds.

### BRIEF DESCRIPTION OF THE DRAWING

FIG. 1 is a cross-sectional view which shows an embodiment of a carrier for cell culture according to the present invention.

### DETAILED DESCRIPTION OF THE INVENTION

In view of the problem described above, the inventors have conducted extensive research and, as a result, found that by forming a carrier for cell culture (hereinafter, simply referred to as a “carrier”) using a calcium phosphate-based compound, which has high compatibility with a variety of kinds of cells, it is possible for cells to satisfactorily adhere to the surface of the carrier and it is also possible to make the carrier suitable for the growth of cells.

In addition, since a calcium phosphate-based compound is biologically inactive, a possibility of damage being caused to cells is extremely low. The present invention has been accomplished on the basis of the findings described above.

Hereinbelow, a detailed description will be made with regard to a preferred embodiment of a carrier according to the present invention with reference to the accompanying drawing.

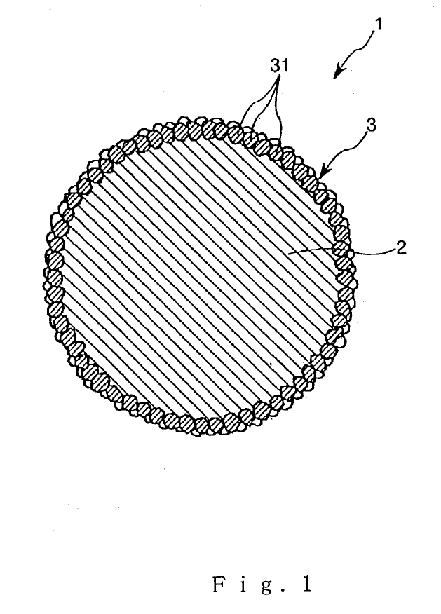

FIG. 1 is a cross-sectional view which shows an embodiment of a carrier according to the present invention. As shown in FIG. 1, the carrier 1 of the present invention comprises a particulate base body 2 which is mainly formed of a resin material and a coating layer 3 of a calcium phosphate-based compound. The base body preferably contains such a resin material of at least 70 to 98% of its weight. Of course, the entire of the base body may be formed of the resin. The coating layer 3 is provided on the surface of the base body 2. By using such a carrier 1, cells (anchorage-dependent cells) are able to properly adhere to and grow on the surface of the carrier. Therefore, the carrier 1 can be used for cell culture, especially high-density three-dimensional cell culture (suspension culture) that achieves a high level of cell density.

Examples of a technique for such high-density three-dimensional cell culture include microcarrier culture, spinner culture, rotary shaking culture, and rotation culture. In particular, among these techniques, the carrier 1 can be suitably used in microcarrier culture. By utilizing microcarrier culture technique, it is possible to culture large quantities of cells highly efficiently.

Microcarrier culture is a technique which makes it possible to grow cells on the surfaces of micro carriers (carriers for cell culture) which are suspended in a culture solution (liquid medium) under gentle agitation. Therefore, a carrier for use in microcarrier culture is required to have various properties (characteristics), for example, a size suitable for cell growth, a specific gravity which makes it possible for carriers to be uniformly suspended in a culture solution, a high strength which prevents a carrier from being broken as a result of agitation, or the like.

The carrier 1 of the present invention satisfies these requirements. Namely, the inventors have found that by coating the surface of the base body 2 mainly formed of a resin material with a calcium phosphate-based compound, the characteristics described above can be imparted easily and reliably to the carrier 1.

For example, the specific gravity (density) of the carrier 1 can be easily adjusted by changing the content of a resin material in the base body 2, or by changing the kind of resin material which is used to form the base body 2.

Since the base body 2 is in a particulate form (preferably in a substantially spherical particulate form), the carrier 1 is also in a particulate form (preferably in a substantially spherical particulate form) as a whole. As a result, cells are easily able to adhere to and grow on the surface of the carrier 1 with high uniformity. Also, the carrier 1 can be uniformly suspended in a culture solution.

The particle size of the carrier 1 is not limited to any specific value, but when the maximum length of a cell (which is to be adhered to the carrier 1) is defined as L1 (μm) and the average particle size of the carrier 1 is defined as L2 (μm), L2/L1 is preferably within the range of 2 to 100, and more preferably within the range of 5 to 50. Specifically, L2 is preferably about 50 to 500 μm, and more preferably about 100 to 300 μm. The size of a cell can be determined, for example, by staining cells removed from the carriers after cultivation and then observing them using an optical microscope. The average particle size of the carrier can be measured, for example, by a particle size analyzer.

By setting the average particle size of the carrier 1 within the above range, the carrier 1 can have a sufficiently large surface area relative to the size of a cell, which enables cells to easily adhere to and grow on the carrier 1. In this regard, if the average particle size of the carrier 1 is too small, not only may cells not easily adhere to the carrier, but also agglomeration may easily occur between the carriers 1. On the other hand, if the average particle size of the carrier 1 is too large, the settling velocity of the carrier 1 in a culture solution increases and it is therefore necessary to increase the speed of agitation (which will be described later) during cell culture. In such a case, collision between the carriers 1 will occur and, as a result, there is a possibility that cells adhered to the surfaces of the carriers 1 will be damaged.

Further, in view of more uniform suspension of the carriers 1 in a culture solution, the density of the carrier 1 is preferably close to that of water. Specifically, the density of the carrier 1 is preferably set to about 0.8 to 1.4 g/cm³, and more preferably set to about 0.9 to 1.2 g/cm³. By setting the density of the carrier 1 to within the above range, it is possible to create a more uniform suspension of the carriers 1 in a culture solution.

The form, size (e.g., average particle size), physical properties (e.g., density) and the like of the carrier 1 can be adjusted by appropriately setting the form, size, physical properties and the like of the base body 2.

As described above, the base body 2 of the carrier 1 is mainly formed of a resin material. By forming the base body 2 using a resin material as a main material, the form, size, physical properties, and the like of the carrier 1 can be easily adjusted.

In manufacturing a carrier 1 having an average particle size as has been described above, the average particle size of the base body 2 is preferably about 50 to 500 μm, and more preferably about 100 to 300 μm.

Further, in manufacturing a carrier 1 having a density as has been described above, the density of the base body 2 is preferably about 0.8 to 1.4 g/cm³, and more preferably about 0.9 to 1.2 g/cm³.

In the present invention, various kinds of thermoplastic resins and various kinds of thermosetting resins can be employed as a resin material which is used for forming the base body 2. Examples of such thermoplastic resins include polyamide, polyethylene, polypropylene, polystyrene, polyimide, acrylic resin, thermoplastic polyurethane and the like; and examples of such thermosetting resins include epoxy resin, phenolic resin, melamine resin, urea resin, unsaturated polyester, alkyd resin, thermosetting polyurethane, ebonite and the like. One kind of these resins or a mixture of two or more kinds of these resins can be employed.

In addition, such resin materials may be colored using organic pigments, inorganic pigments, acid dyes, basic dyes and the like.

As described above, there is provided the coating layer 3 of a calcium phosphate-based compound on the surface of the base body 2. The coating layer 3 is formed from particles 31 of a calcium phosphate-based compound, and the particles 31 on the surface are partially embedded in the base body at the vicinity of the surface thereof (that is, the particles 31 are partially embedded in a surface area including and adjacent to the surface of the base body 2), thereby coating the surface of the base body 2 with a calcium phosphate-based compound.

By forming the coating layer 3 in this way, excellent adhesion is provided between the coating layer 3 and the base body 2, thereby preventing detachment of the coating layer 3 from the surface of the base body 2. Namely, it is possible to obtain a carrier in which the base body 2 is reliably coated with a calcium phosphate-based compound.

The average thickness of the coating layer 3 is not limited to any specific value, but is preferably about 0.1 to 5 μm, and more preferably about 0.5 to 2 μm. If the average thickness of the coating layer 3 is less than the above lower limit value, there is a case that a part of the surface of the base body 2 is exposed in the carrier 1. On the other hand, if the average thickness of the coating layer 3 exceeds the above upper limit value, it becomes difficult to adjust the density of the carrier 1 to within the range described above.

The surface area of the coating layer 3 (carrier 1) may be either dense or porous.

The kind of calcium phosphate-based compound that can be used in the present invention is not particularly limited, and various kinds of compounds having a Ca/P ratio of 1.0 to 2.0 can be used. Examples of such compounds include Ca₁₀(PO₄)₆(OH)₂, Ca₁₀(PO₄)₆F₂, Ca₁₀(PO₄)₆Cl₂, Ca₃(PO₄)₂, Ca₂P₂O₇, Ca(PO₃)₂, CaHPO₄, and the like, and one kind of these compounds or a mixture of two or more kinds of these compounds can be employed.

Among these compounds, a calcium phosphate-based compound containing hydroxyapatite (Ca₁₀(PO₄)₆(OH)₂) as a main component is most suitable. Since hydroxyapatite is used as a biomaterial, a carrier having a coating layer formed of hydroxyapatite is unlikely to cause damage to cells, and cells can highly efficiently adhere to such a carrier.

Further, in a case that fluorapatite (Ca₁₀(PO₄)₆F₂) is used, it is preferred that the percentage of fluorine content in the entire calcium phosphate-based compound is 5 wt % or less. By setting the percentage of fluorine content in the entire calcium phosphate-based compound to 5 wt % or less, it is possible to prevent or minimize the elution of fluorine from the coating layer 3 (carrier 1). Therefore, a possibility that the carrier 1 causes damage to cells is eliminated or minimized and, as a result, the growth efficiency of cells is prevented from being decreased.

It is to be noted that the calcium phosphate-based compounds described above can be synthesized by means of a wet method, a dry method or the like, which are well known in the art.

In such a method, a synthesized calcium phosphate-based compound may include residual substances (e.g., raw materials) and/or secondary products, which are produced in synthesis.

The coating layer 3 can be formed by, for example, colliding porous particles of a calcium phosphate-based compound (hereinafter, simply referred to as “porous particles”) against the surface of the base body 2. By such a method, it is possible to form the coating layer 3 easily and reliably.

By colliding the porous particles against the surface of the base body 2, they are broken into particles 31 having a relatively small particle size (hereinafter, simply referred to as a “particle 31”) when collided against the base body 2, and the particles 31 on the surface are partially embedded in the base body 2. The base body 2 captures the particles 31 using an elastic force thereof, thereby securing the particles 31 on the base body 2.

Further, the porous particle is preferably produced by agglomerating primary particles of a calcium phosphate-based compound. By using such porous particles, it is possible to more reliably coat the surface of the base body 2 because such porous particles are more effectively fragmented when collided against the base body 2.

The average particle size of the porous particle is not limited to any specific value, but is preferably 100 μm or less. If the average particle size of the porous particle exceeds 100 μm, there is a case that the velocity of the porous particle at the time of collision against the base body 2 will be too low, and the porous particle will not be effectively fragmented.

Collision between the base bodies 2 and the porous particles can be carried out, for example, by using a hybridization machine (commercially available) in a dry condition. In this case, conditions may be set such that, for example, the mixing ratio of the base bodies 2 and the porous particles is about 400:1 to 50:1 in weight ratio, and a temperature within the hybridization machine is equal to or less than a softening temperature of a resin material which is used as a main material of the base body 2 (usually 80° C. or less).

Also, the porous particles to be used for forming the coating layer 3 can be produced, for example, in a manner well known in the art, as will be described below.

First, a calcium phosphate-based compound is synthesized by a well known wet method to obtain a slurry in which crystalline particles (primary particles) of the synthesized calcium phosphate-based compound (initial material) are suspended. Then, the slurry is directly spray-dried to thereby obtain granulated secondary particles. Alternatively, such secondary particles may be obtained by adding an additive such as a viscosity adjusting agent, particles of an organic compound or fibers which can be evaporated by heating, or the like to the slurry and then spray-drying the slurry. It is to be noted that the thus obtained secondary particles may be sintered as needed.

Since the thus obtained secondary particles are porous, such secondary particles can be directly used in forming the coating layer 3.

In a case that it is preferred that porous particles having higher porosity are used, such porous particles are produced, for example, in a manner as will be described below.

First, a slurry is prepared in which the secondary particles obtained in the above-described manner are suspended, and then the slurry is formed into a block shape by wet pressing, dry pressing or the like. In this regard, it is to be noted that an organic compound which can be evaporated in the following sintering process to provide pores may be added to the slurry. The diameter of pores may also be controlled by adjusting a condition such as a sintering temperature or the like instead of addition of such an organic compound as has been described above. Then, the thus obtained block is sintered at a temperature within the range of 400 to 1,300° C. If the sintering temperature is less than 400° C., there may be a case that the added organic compound will not be fully evaporated, or the block will not be satisfactorily sintered. On the other hand, if the sintering temperature exceeds 1,300° C., there may be a case that a resulting sintered body will be excessively dense, or the calcium phosphate-based compound will be decomposed. Thereafter, the thus sintered block is ground and then classified to obtain particles having a desired particle size.

The diameter of pores in the porous particle can be adjusted, for example, by appropriately setting the size of the primary particle, the viscosity of the slurry, the kind of additive, and the like. It is to be noted that the diameter of pores in the porous particle is preferably 500 to 1,000 Å, and the specific surface area of the porous particle is preferably 10 m²/g or more. By using such porous particles for manufacturing a carrier 1, it is possible for cells to adhere to and grow on the carrier 1 more efficiently.

It is to be noted that while a method for forming the coating layer 3 (that is a method for manufacturing the carrier 1) has been described above, the present invention is not limited thereto.

Hereinbelow, a description will be made with regard to a method for culturing cells according to the present invention, that is, a method for culturing cells using the carriers 1 described above.

<1> First, the carriers 1 are subjected to sterilization, to thereby decrease the number of microorganisms or molds existing on the surfaces of the carriers 1 or destroy all such microorganisms or molds. By subjecting the carriers 1 to sterilization prior to use, a possibility that microorganisms or molds cause damage to cells is decreased or eliminated, thereby enabling cells to more efficiently grow on the surfaces of the carriers 1.

Examples of a sterilization technique include sterilization using a sterilizing solution, autoclave sterilization, gaseous sterilization, radiation sterilization, or the like. Among these techniques, sterilization using a sterilizing solution is preferably used, which is performed by immersing or contacting carriers in or to a sterilizing solution. By such a technique, it is possible to more efficiently sterilize large quantities of the carriers 1.

It is to be noted here that deterioration of the carrier 1 of the present invention upon immersion in (contact to) a sterilizing solution is prevented, because the carrier 1 has the coating layer 3 made of a calcium phosphate-based compound that is inert to various kinds of sterilizing solutions. Therefore, the carrier 1 of the present invention is suitable for sterilization using a sterilizing solution.

As for a sterilizing solution, an alkaline solution such as an aqueous solution of sodium hydroxide, an aqueous solution of potassium hydroxide, an aqueous solution of sodium hypochlorite or the like is preferably used. Such sterilizing solutions have especially excellent sterilizing properties for destroying (reducing) microorganisms or molds.

Following sterilization, the carriers 1 are rinsed to remove a sterilizing solution from the surfaces of the carriers 1.

<2> Next, a culture solution is prepared in which the carriers 1 sterilized in the process <1> and cells (which are to be adhered to the carrier) are suspended.

In this regard, it is to be noted that, for example, a shuttle vector containing a protein-coding gene has been previously introduced into the cell so that a target protein can be produced.

Examples of the cell include an animal cell, a plant cell, a bacterium, a virus and the like. Among them, an animal cell is especially preferred. An animal cell can be applied in various fields, and by using animal cells it is possible to effectively produce a protein having a complex structure (e.g., glycoprotein).

The kind of culture solution can be appropriately selected depending on the kind of cell to be used, and is not limited to any specific one. Examples of the culture solution include Dulbecco's MEM (Dulbecco's Modified Eagle's Medium), BME (Eagle's Basal Medium), MCDB-104 medium, and the like.

Further, an additive such as serum, serum protein (e.g., albumin), various kinds of vitamins, amino acids or salts, and the like may be added to the culture solution as required.

Then, the prepared culture solution is agitated to cause the cells to adhere to the surfaces of the carriers 1, and the cells adhered to the surfaces grow on the carriers 1 over time. In this way, the cells are cultured. Agitation of the culture solution increases cell growth efficiency in culturing cells.

The speed of agitation of the culture solution is not limited to any specific value, but is preferably set to about 5 to 100 rpm, and more preferably set to about 10 to 50 rpm. If the speed of agitation is too low, there is a case that the carriers 1 are not uniformly dispersed in the culture solution depending on the density, average particle size or the like of the carrier 1, as a result of which the cells will not be able to satisfactorily grow on the surfaces of the carriers 1. On the other hand, if the speed of agitation is too high, there is a case that the carriers 1 will be subjected to excessive agitation resulting in violent collision between the carriers 1, thereby causing damage to the cells adhered to the carriers.

The temperature (incubation temperature) of the culture solution is appropriately set depending on the kind of cell to be cultured, and is not limited to any specific value, but it is normally set to about 20 to 40° C., and preferably set to about 25 to 37° C.

The grown cells produce a target protein, and the produced protein is released into the culture solution or accumulated within the cells.

<3> Next, the produced protein is collected. For example, the protein released into the culture solution can be collected in a manner as will be described below. First, agitation of the culture solution is stopped to precipitate the carriers 1 in the culture solution. Then, a supernatant liquid is removed. By treating the supernatant liquid (e.g., chromatography), it is possible to easily collect the produced protein.

As described above, the density of the carrier 1 is close to that of water. The density of the carrier 1 is gradually increased overall due to adhesion and growth of cells and, as a result, the carrier 1 can be easily precipitated in the culture solution.

Hereinbelow, a description will be made with regard to actual examples according to the present invention.

### EXAMPLE 1

First, 50 g of nylon beads (base body) having an average particle size of 150 μm and a density of 1.02 g/cm³, and 0.25 g of hydroxyapatite particles (which are porous particles formed by the agglomeration of primary particles) having an average particle size of 10 μm and a Ca/P ratio of 1.67 were prepared. It is to be noted here that the specific surface area of the hydroxyapatite particle was 10 m²/g or more, and the diameter of pores in the hydroxyapatite particle was about 500 to 1,000 Å.

Next, the nylon beads and the hydroxyapatite particles were fed into a NARA HYBRIDIZATION SYSTEM NHS-1 (manufactured by Nara Machinery Co., Ltd. and having a rated power of 5.5 kW and a rated current of 23 A), and the system was then operated at 6,400 rpm and at a temperature within the range of 32 to 50° C. for 5 minutes, by which the nylon beads were coated with hydroxyapatite. In this way, carriers coated with hydroxyapatite were obtained.

The thus obtained carrier had an average particle size of 151 μm (the average thickness of a coating layer of hydroxyapatite was 1 μm) and a density of 1.03 g/cm³.

### EXAMPLE 2

First, 50 g of polystyrene beads (base body) having an average particle size of 450 μm and a density of 1.04 g/cm³, and 0.2 g of hydroxyapatite particles (which are porous particles formed by the agglomeration of primary particles) having a Ca/P ratio of 1.67 and an average particle size of 100 μm were prepared. It is to be noted here that the specific surface area of the hydroxyapatite particle was 10 m²/g or more, and the diameter of pores in the hydroxyapatite particle was about 500 to 1,000 Å.

Next, the polystyrene beads and the hydroxyapatite particles were fed into a NARA HYBRIDIZATION SYSTEM NHS-1 (manufactured by Nara Machinery Co., Ltd. and having a rated power of 5.5 kW and a rated current of 23 A), and the system was then operated at 8,000 rpm and at a temperature within the range of 36 to 64° C. for 5 minutes, by which the polystyrene beads were coated with hydroxyapatite. In this way, carriers coated with hydroxyapatite were obtained.

The thus obtained carrier had an average particle size of 452 μm (the average thickness of a coating layer of hydroxyapatite was 1.5 μm) and a density of 1.05 g/cm³.

### EXAMPLE 3

First, 400 g of polyethylene beads (base body) having an average particle size of 50 μm and a density of 0.92 g/cm³, and 4 g of calcium phosphate particles having a Ca/P ratio of 1.8 and an average particle size of 80 μm (which are porous particles formed by the agglomeration of primary particles) were prepared. It is to be noted here that the specific surface area of the calcium phosphate particle was 10 m²/g or more, and the diameter of pores in the calcium phosphate particle was about 500 to 1,000 Å.

Next, the polyethylene beads and the calcium phosphate particles were fed into a mixer (manufactured by NISSHIN ENGINEERING INC. with a product code of Hi-X200), and the mixer was then operated at a temperature within the range of 25 to 75° C. for 20 minutes with a standard impeller being rotated at 4,000 rpm, by which the polyethylene beads were coated with calcium phosphate. In this way, carriers coated with calcium phosphate were obtained.

The thus obtained carrier had an average particle size of 51 μm (the average thickness of a coating layer of calcium phosphate was 1 μm) and a density of 0.94 g/cm³.

### EXAMPLE 4

First, 50 g of polymethyl methacrylate beads (base body) having an average particle size of 200 μm and a density of 1.19 g/cm³, and 0.2 g of tricalcium phosphate particles (which are porous particles formed by the agglomeration of primary particles) having a Ca/P ratio of 1.5 and an average particle size of 20 μm were prepared. It is to be noted here that the specific surface area of the tricalcium phosphate particle was 10 m²/g or more, and the diameter of pores in the tricalcium phosphate particle was about 500 to 1,000 Å.

Next, the polymethyl methacrylate beads and the tricalcium phosphate particles were fed into a NARA HYBRIDIZATION SYSTEM NHS-1 (manufactured by Nara Machinery Co., Ltd. and having a rated power of 5.5 kW and a rated current of 23 A), and the system was then operated at 8,000 rpm and at a temperature within the range of 38 to 71° C. for 5 minutes, by which the polymethyl methacrylate beads were coated with tricalcium phosphate. In this way, carriers coated with tricalcium phosphate were obtained.

The thus obtained carrier had an average particle size of 204 μm (the average thickness of a coating layer of tricalcium phosphate was 3 μm) and a density of 1.2 g/cm³.

### Comparative Example

Nylon beads having an average particle size of 150 μm and a density of 1.02 g/cm³ were prepared as carriers of Comparative Example.

<Evaluation>

Prior to an evaluation test I and an evaluation test II, the carriers manufactured in each of the Examples 1 to 4 were subjected to autoclave sterilization, ethylene oxide gaseous sterilization, radiation sterilization, and sterilization using an aqueous solution of sodium hydroxide (an alkaline solution), respectively. The carriers prepared in Comparative Example were subjected to ethylene oxide gaseous sterilization.

<Evaluation Test I>

For the carriers manufactured in each of the Examples 1 to 4 and Comparative Example, an evaluation test I was carried out in a manner as will be described below.

1 g of the carriers and 10 ml of a suspension containing 1×10⁵ human osteosarcoma-derived cells (Saos2) per milliliter were added to 100 ml of Dulbecco's MEM (culture solution).

In this regard, it is to be noted that 10 vol % of fetal bovine serum had been previously added to the Dulbecco's MEM.

Human osteosarcoma-derived cells (Saos2) were cultured with the Dulbecco's MEM being agitated at 30 rpm and at a temperature of 37° C. for 3 hours. It is to be noted that the maximum length of the human osteosarcoma-derived cell is about 20 μm.

A predetermined amount of the culture solution was sampled every 60 minutes until 180 minutes have passed from the beginning of cultivation (beginning of agitation) to count cells adhered to the surfaces of the carriers.

In this regard, it is to be noted that counting of cells was carried out using trypan blue staining method in which trypan blue is added to trypsin-treated cells. The result of counting of cells is shown in Table 1.

<Evaluation Test II>

For the carriers manufactured in each of the Examples 1 to 4 and Comparative Example, an evaluation test II was carried out in a manner as will be described below.

1 g of the carriers and 10 ml of a suspension containing 1×10⁵ mouse calvaria-derived cells (MC3T3E1) per milliliter were added to 100 ml of Dulbecco's MEM (culture solution).

In this regard, it is to be noted that 10 vol % of fetal bovine serum had been previously added to the Dulbecco's MEM.

Mouse calvaria-derived cells (MC3T3E1) were cultured with the Dulbecco's MEM being agitated at 30 rpm and at a temperature of 37° C. for 3 hours. It is to be noted that the maximum length of the mouse calvaria-derived cell is about 20 μm.

A predetermined amount of the culture solution was sampled every 60 minutes until 180 minutes have passed from the beginning of cultivation (beginning of agitation) to count cells adhered to the surfaces of the carriers.

In this regard, it is to be noted that counting of cells was carried out using trypan blue staining method in which trypan blue is added to trypsin-treated cells. The result of counting of cells is shown in Table 1.

TABLE 1
Number of Cells (10⁵ cells)

| | | after 60 | after 120 | after 180 |
|---|---|---|---|---|
| | Kind of Cell | minutes | minutes | minutes |
| Example 1 | Human | 4.6 | 7.5 | 8.0 |
| Example 2 | Osteosarcoma- | 4.5 | 7.0 | 7.8 |
| Example 3 | derived Cell | 2.5 | 4.0 | 4.5 |
| Example 4 | (Saos2) | 4.2 | 5.5 | 6.9 |
| Comparative | | 2.2 | 2.8 | 3.1 |
| Example | | | | |
| Example 1 | Mouse | 6.0 | 7.7 | 8.5 |
| Example 2 | Calvaria- | 5.3 | 8.0 | 8.1 |
| Example 3 | derived Cell | 2.6 | 4.2 | 6.3 |
| Example 4 | (MC3T3E1) | 4.7 | 6.8 | 7.2 |
| Comparative | | 2.5 | 2.7 | 2.8 |
| Example | | | | |

As shown in Table 1, in both the evaluation tests I and II where human osteosarcoma-derived cells and mouse calvaria-derived cells were cultured, respectively, the number of cells adhered to the carriers manufactured in each of the Examples 1 to 4 (carriers of the present invention) was larger than the number of cells adhered to the carriers manufactured in Comparative Example in absolute number at the time when 60 minutes have passed from the beginning of cultivation. This means that the carriers manufactured in each of the Examples 1 to 4 are suitable for adhesion of cells. Also, at the time when 120 minutes and 180 minutes have passed from the beginning of cultivation, the number of cells adhered to the carriers manufactured in each of the Examples 1 to 4 was larger than the number of cells adhered to the carriers manufactured in Comparative Example, respectively. Further, the cells adhered to the carriers manufactured in each of the Examples 1 to 4 efficiently grew as compared to the cells adhered to the carriers manufactured in Comparative Example.

Namely, it has been confirmed that by using any of the carriers manufactured in Examples 1 to 4 (carriers of the present invention), it is possible for cells to efficiently adhere to and grow on the carriers irrespective of the kind of cell. On the other hand, it is apparent that use of the carriers manufactured in Comparative Example results in extremely poor adhesion of cells and significant reduction in cell growth efficiency.

As has been described above, according to the present invention, it is possible to obtain a carrier having a variety of properties that are required for a carrier for cell culture (especially, microcarrier culture). Also, the carrier of the present invention can be manufactured at low cost.

Finally, it is to be understood that many changes and additions may be made to the embodiments described above without departing from the scope and spirit of the invention as defined in the following claims.

Further, it is also to be understood that the present disclosure relates to subject matter contained in Japanese Patent Application No.2002-048603 (filed on Feb. 25, 2002) which is expressly incorporated herein by reference in its entireties.

What is claimed is:

1. A carrier for cell culture which enables cells to adhere to and grow on a surface thereof, the carrier comprising:
a base body having a particulate form and being mainly formed of a resin material, the base body having a surface; and
a coating layer formed from particles of a calcium phosphate-based compound, wherein the coating layer is provided on the surface of the base body, with the particles of the calcium phosphate-based compound being partially embedded in the base body at the vicinity of the surface thereof.

2. The carrier for cell culture as claimed in claim 1, wherein when the maximum length of the cell to be adhered to the carrier for cell culture is defined as L1 (μm), and the average particle size of the carrier for cell culture is defined as L2 (μm) , L2/L1 is within the range of 2 to 100.

3. The carrier for cell culture as claimed in claim 2, wherein L2 is within the range of 50 to 500 μm.

4. The carrier for cell culture as claimed in claim 1, wherein the density of the carrier for cell culture is within the range of 0.8 to 1.4 g/cm³.

5. The carrier for cell culture as claimed in claim 1, wherein the average particle size of the base body is within the range of 50 to 500 μm.

6. The carrier for cell culture as claimed in claim 1, wherein the density of the base body is within the range of 0.8 to 1.4 g/cm³.

7. The carrier for cell culture as claimed in claim 1, wherein the average thickness of the coating layer is within the range of 0.1 to 5 μm.

8. The carrier for cell culture as claimed in claim 1, wherein the coating layer is formed by colliding porous particles of the calcium phosphate-based compound against the surface of the base body.

9. The carrier for cell culture as claimed in claim 8, wherein the porous particles are manufactured by agglomerating primary particles of the calcium phosphate-based compound.

10. The carrier for cell culture as claimed in claim 1, wherein the carrier for cell culture is used in microcarrier culture.

11. The carrier for cell culture as claimed in claim 1, wherein the cell is an animal cell.

12. A method for culturing cells, the method being characterized by using carriers containing the carrier for cell culture claimed in claim 1.

13. The method for culturing cells as claimed in claim 12, wherein the carriers have been subjected to sterilization.

14. The method for culturing cells as claimed in claim 13, wherein the sterilization of the carriers is carried out using a sterilizing solution.

15. The method for culturing cells as claimed in claim 14, wherein the sterilizing solution is an alkaline solution.

16. A method for culturing cells, the method being characterized in that a culture solution, in which carriers containing the carrier for cell culture claimed in claim 1 and cells are suspended, is agitated to cause the cells to adhere to the surfaces of the carriers, thereby enabling the cells to grow thereon.

17. The method for culturing cells as claimed in claim 16, wherein the speed of agitation is within the range of 5 to 100 rpm.

18. The method for culturing cells as claimed in claim 16, wherein the carriers have been subjected to sterilization.

19. The method for culturing cells as claimed in claim 18, wherein the sterilization of the carriers is carried out using a sterilizing solution.

20. The method for culturing cells as claimed in claim 19, wherein the sterilizing solution is an alkaline solution.

21. A microcarrier culture comprising the carrier for cell culture recited in claim 1.

22. A microcarrier culture comprising the carrier for cell culture recited in claim 2.

23. A microcarrier culture comprising the carrier for cell culture recited in claim 5.
